SEQUENCE LISTING (4 sequences)

SEQ ID NO: 1 - DNA, 24 bp, Artificial Sequence
  CDK 5 Primer 1
ggggatgatgagggtgttccaagc

SEQ ID NO: 2 - DNA, 25 bp, Artificial Sequence
  CDK 5 Primer 2
ggcgaccggcatttgagatctctgc

SEQ ID NO: 3 - DNA, 23 bp, Artificial Sequence
  TOR-2 Primer 1
caattatcatgcgttatacaaag

SEQ ID NO: 4 - DNA, 22 bp, Artificial Sequence
  TOR-2 Primer 2
cattccacttcgataagtattg

(19) United States
(12) Patent Application Publication
(10) Pub. No.: US 2011/0064721 A1
(43) Pub. Date: Mar. 17, 2011

(54) CATHEPSIN-D NEUROPROTECTION

(75) Inventors: Jianhua Zhang, Birmingham, AL (US); Guy Caldwell, Northport, AL (US); Kevin Roth, Birmingham, AL (US); David Standaert, Birmingham, AL (US); Kim Cladwell, Northport, AL (US); Shusei Hamamichi, Tuscaloosa, AL (US)

(73) Assignees: THE UAB RESEARCH FOUNDATION, Birmingham, AL (US); THE UNIVERSITY OF ALABAMA, Tuscaloosa, AL (US)

(21) Appl. No.: 12/667,832

(22) PCT Filed: Jul. 2, 2008
(86) PCT No.: PCT/US08/69041

§ 371 Date: Sep. 9, 2010

Related U.S. Application Data
(60) Provisional application No. 60/949,457, filed on Jul. 12, 2007.

(51) Int. Cl. A61K 39/395; C12Q 1/37; A61K 38/02; A61P 25/28; A61P 25/16; A61K 31/7088; C12Q 1/68; A61K 38/48

(52) U.S. Cl. 424/130.1; 435/23; 514/17.8; 514/44 R; 435/6; 424/94.66; 514/17.7

(57) ABSTRACT

Provided herein are methods and compositions for promoting neuroprotection in a subject and for treating a neural disorder associated with protein aggregation comprising administering to the subject an agent that increases expression or activity of cathepsin-D. Also provided are methods of screening for agents that increase expression or activity of cathepsin-D and methods of screening for neuroprotective agents.

### CROSS-REFERENCE TO PRIORITY APPLICATIONS

This application claims priority to U.S. Provisional Application No. 60/949,457, filed Jul. 12, 2007, which is incorporated herein by reference in its entirety.

### BACKGROUND

Patients with α-syn A53T, A30P, E46K mutations or gene amplification develop typical Parkinson's disease (PD) and often an associated dementia. However, in >90% of PD cases, and almost all Dementia with Lewy bodies (DLB) and Alzheimer's Disease (AD) cases, there is no clear evidence for mutation or overproduction of α-syn. Therefore, impaired α-syn clearance may play a more important role than α-syn overexpression in neuronal α-syn accumulation and disease pathogenesis.

Experiments in vitro have shown that α-syn can be cleared by the cytosolic ubiquitin-proteasome system (UPS), and/or lysosome-mediated autophagic pathways.

The UPS degrades short-lived, misfolded and/or damaged proteins via an ubiquitin-dependent signaling pathway. Macroautophagy is initiated by de novo synthesis of double membrane vesicles in the cytoplasm. These vesicles encircle long-lived or damaged proteins or organelles by an unknown signaling mechanism and deliver these cargos to lysosomes for degradation. Chaperone-mediated autophagy (CMA) is initiated by chaperones binding to cytosolic proteins followed by delivery to the lysosomes via LAMP-2a receptors. Wildtype α-syn has a pentapeptide sequence that can serve as a CMA recognition motif and can be translocated to the lysosome, while pathogenic A53T and A30P mutant α-syn block CMA.

Lysosomal function declines with age in the human brain. Accumulation of autophagic vacuoles (AVs) has been reproducibly observed in postmortem AD and PD patient brains compared to normal controls, consistent with either an overproduction of AVs or a deficit in autophagolysosomal clearance. Enhancing macroautophagy by either mTOR-dependent or independent mechanisms can help clear aggregation-prone proteins, including huntingtin, A53T and A30P mutant α-syn. However, because both macroautophagy and CMA are dependent on intact lysosomes, if lysosomal function is impaired, enhancing macroautophagy may not be effective in clearing potentially neurotoxic proteins.

### SUMMARY

Provided herein are methods and compositions for promoting neuroprotection in a subject and for treating a neural disorder associated with protein aggregation. For example, methods for promoting neuroprotection or for treating a neural disorder associated with protein aggregation in a subject comprise administering to the subject an agent that increases expression or activity of cathepsin-D.

Also provided are methods of screening for agents that increase expression or activity of cathepsin-D (CD) and methods of screening for neuroprotective agents. For example, provided is a method of screening for agents that increase expression or activity of cathepsin-D comprising contacting a cell with an agent to be tested and determining the level of expression or activity of cathepsin-D, wherein an increase in the expression or activity of cathepsin-D as compared to a control indicates that the agent increases expression or activity of cathepsin-D. Also provided is a method of screening for agents that increase expression or activity of cathepsin-D in a subject comprising administering an agent to be tested to the subject and determining the level of expression or activity of cathepsin-D in the subject, wherein an increase in the expression or activity of cathepsin-D as compared to a control indicates that the agent increases expression or activity of cathepsin-D. A method of screening for neuroprotective agents is also provided, comprising contacting a cell with an agent to be tested and determining the level of expression or activity of cathepsin-D, wherein an increase in the expression or activity of cathepsin-D as compared to a control indicates that the agent is a neuroprotective agent. Also provided is a method of screening for neuroprotective agents in a subject, comprising administering an agent to be tested to the subject and determining the level of expression or activity of cathepsin-D in the subject, wherein an increase in the expression or activity of cathepsin-D as compared to a control indicates that the agent is a neuroprotective agent.

### DESCRIPTION OF DRAWINGS

FIG. 1A shows immunohistochemistry analysis with anti-α-syn and anti-ubiquitin antibodies by DAB staining in p25 CD+/+ (+/+) and CD−/− (−/−) cortex. Scale bar=20 micron. Arrows point to the aggregates. FIG. 1B shows α-syn aggregates in NeuN+neuronal cell bodies using immunofluorescence. Wild-type (+/+) brains exhibit diffuse α-syn staining. CD−/− brains showed neurons with cytoplasmic accumulation of α-syn immunoreactivity. Scale bar=10 micron. FIG. 1C is an immunofluorescence micrograph showing α-syn did not aggregate in GFAP+cells. Scale bar=10 micron. FIG. 1D is a Western blot showing accumulation of high molecular weight α-syn and ubiquitinated proteins in both the TritonX100 soluble and the insoluble fractions of the CD−/− mice. Intensity of α-syn monomer, α-syn oligomers, and Ub-positive smears were quantified and compared between CD+/+ and CD−/− extracts. Truncated 12 kd and 10 kd α-syn fragments were reduced in CD−/− extracts. FIG. 1E is a graph showing quantification of the Western blot results from FIG. 1D. N=3 mice each genotype. *p<0.05 compared to CD+/+ by Student t-test. S=TritonX100 soluble. IS=TritonX100 insoluble.

FIGS. 2A-2C are immunofluorescence micrographs showing α-syn aggregates are adjacent to, but not overlapping with, LC3 or CB, and are not in neurons with active caspase-3 immunoreactivity. FIG. 2A is an immunostaining with LC3/ATG8 and α-syn antibodies showing that LC3 staining was increased in CD−/− mice compared to CD+/+ mice, and partially overlapped with α-syn aggregates (p25). Arrows point to α-syn aggregates adjacent to LC3 staining. Arrowheads point to cells with high LC3 staining but no α-syn aggregate. FIG. 2B shows α-syn aggregates were adjacent to but do not appear to overlap with CB. Arrow points to α-syn aggregates adjacent to CB staining. FIG. 2C shows neurons with positive active caspase-3 staining did not exhibit intense α-syn aggregates in CD−/− brains. Arrows point to active caspase-3 immunoreactivity. Scale bar=10 micron. n=3 mice each genotype.

FIG. 3A is a graph showing α-syn mRNA was down-regulated in CD deficient brains compared to wildtype control brains. CB, CL, CF, CH, Atg7, UCHL1, Parkin, and UPS β2, mRNA levels were up-regulated. Atg12 and UPS β1 mRNA levels were normal. FIG. 3B is a Western blot analysis with a bar graph showing an increase of steady state GAPDH, a CMA substrate. N=3 p25 brain. * p<0.05 by Student t-test, compared between wild-type (+/+) and CD−/− brains. FIG. 3C is a bar graph showing extracts from CD−/− cortex exhibited reduced proteasome activities compared to CD+/+ as indicated by assays with trypsin-like fluorigenic substrate (VGR-AMC, reaching maximum at 60 min), chymotrypsin-like fluorigenic substrate (Z-GGL-AMC, reaching maximum at 120 min), and peptidylglutamyl peptide-like flurorigenic substrate (Suc-LLVY-AMC, reaching maximum at 120 min). The activities that were inhibited by the proteasome inhibitor lactacystin were quantified. n=3 mice each genotype. *p<0.05 by Student t-test. FIG. 3D is a Western blot showing normal expression of proteins involved in UPS. Western blot analyses of UCHL1, Usp14, Rpt3, α4 and β1 show that these UPS factors were expressed normally in CD+/+ and CD−/− cortical extracts. Actin immunoblotting was used as a loading control. n=3 mice each genotype.

FIG. 4A is a bar graph showing CD reduced α-syn aggregation in an aggregation assay. 50% cells transfected with α-syn-GFP, synphilin and empty vector exhibited visible α-syn aggregates. Co-transfection of CD together with α-syn-GFP and synphilin reduced the number of cells with visible α-syn aggregates to 20%. N=3 independent transfection, each in quadruplicate. *p<0.05 compared to absence of exogenous CD by Student t-test. FIG. 4B is a bar graph showing enhanced CD expression protected against α-syn overexpression-induced cell death. GFP was visualized under the fluorescence microscope and demonstrated more survival cells after co-transfection of GFP-α-syn and CD compared to transfection with GFP-α-syn alone. Viable cells were counted by trypan blue exclusion method. FIG. 4C shows a Western blot analysis and a bar graph indicating that CD transfection resulted in truncation of α-syn-GFP and a reduction of endogenous α-syn monomers. FIG. 4D is a bar graph showing enhanced CD expression reduced A53T and A30P mutant α-syn-induced cell death, but does not reduce Y125A mutant α-syn-, 10 μM chloroquine-, or 2 μM staurosporine-induced cell death. For FIGS. 4B-4D, *p<0.05 compared to control (CTL); †p<0.05 compared to otherwise identical transfection except without CD. n=3 transfection for each experimental conditions. Student t-test was used.

FIGS. 5A-5F are immunofluorescence micrographs showing RNAi knockdown of a C. elegans CD ortholog worsens aggregation of human α-syn in vivo. Isogenic worm strains expressing α-syn::GFP alone (FIG. 5A) or with TOR-2 (FIG. 5B) in the body wall muscle cells of C. elegans were examined. See the Examples below. The presence of TOR-2, a protein with chaperone activity, attenuated the misfolded α-syn protein (FIG. 5B). When worms expressing α-syn::GFP+TOR-2 were exposed to CD RNAi, the misfolded α-syn::GFP returned (FIG. 5C). FIGS. 5D and 5E are immunoflourescence micrographs and FIG. 5F is a bar graph showing overexpression of CD protected dopamine (DA) neurons from α-syn-induced degeneration. Worm DA neurons degenerated as animals age. At the 7-day stage, most worms were missing anterior DA neurons of the CEP (cephalic) and/or ADE (anterior deirid) classes. For FIG. 5D, note the presence of 3 of 4 CEP DA neurons (arrows) and the absence of the 2 ADE neurons. FIG. 5E shows overexpression of CD protects worms from neurodegeneration whereby worms displayed all 4 CEP (arrows) and both ADE (arrowheads) neurons. FIG. 5F shows the percentage of worms exhibiting the wildtype neuronal complement of all 6 anterior DA neurons (30%) was significantly greater than animals without CD overexpression (15%). CD mutants (D295R and F2291), CB and CL, in transgenic worms overexpressing human cDNAs encoding these mutated CD or the representative lysosomal cysteine proteases, did not have the same effect as the wildtype CD in reducing α-syn toxicity. *p<0.001 compared to α-syn alone, by Fisher Exact Test.

FIG. 6 are fluorescence micrographs showing AAV-CD delivered and allowed expression of CD in the SNr of mice. A CD expressing construct using rAAV was created, which co-expresses CD and EGFP under CMV promoter. AAV-CD was injected using stereotaxic method in unilateral SNr region at 3 months of age. After injection, the mice were perfused and immunohistochemistry studies were performed for the expression and localization of tyrosine hydroxylase positive (TH+) neurons and CD.

### DETAILED DESCRIPTION

Cathepsin-D (CD) is the principal lysosomal aspartate protease and a main endopeptidase responsible for the degradation of long-lived proteins, including α-syn. CD is expressed widely in the brain, including in the cortex, hippocampus, striatum, and dopaminergic neurons of the substantia nigra (SNr). CD is synthesized as a precursor with a signal peptide cleaved upon its insertion into endoplasmic reticulum. The CD zymogen is activated in an acidic environment by cleavage of the pro-peptide.

CD homozygous inactivation was reported to cause human congenital neuronal ceroid lipofuscinosis (NCL) with postnatal respiratory insufficiency, status epilepticus, and death within hours to weeks after birth. These patients had severe neurological defects in early childhood and alterations in α-syn accumulation had not yet been reported. Another patient with significant loss of CD enzymatic function (7.7% Vmax from patient fibroblast lysates compared to controls) due to compound heterozygous missense mutations developed childhood motor and visual disturbances, cerebral and cerebellar atrophy, and progressive psychomotor disability. Milder forms of CD deficiency predispose one to late onset neurodegenerative disorders, including AD and PD. Parkinsonism has been noted in lysosomal tripeptidyl peptidase I deficient patients, adult forms of NCL patients, and Gaucher disease patients. α-syn aggregation has been reported in both neurons and glia in several lysosomal disorders, such as Gaucher disease, Niemann-Pick disease, GM2 gangliosidosis, Tay-Sachs, Sandhoff disease, metachromatic leukodystrophy, and beta-galactosialidosis.

Significant increase in α-syn aggregates has not been previously reported in mouse models of proteolytic disorders involving proteasomes, autophagy or other lysosomal proteases. As described herein, a robust α-synucleinopathy in CD deficient mice was observed, despite the compensatory up-regulation of other lysosomal proteases, and the presence of normal wildtype levels of α-syn mRNA expression. As described herein, it was observed that proteasome activities are significantly reduced in the CD-deficient brain, whereas several key UPS factors are either normal or up-regulated, indicating crosstalk between lysosomal and proteasomal activities at the levels of signaling rather than a reduction of protein levels. Finally, as described in the examples below CD, but not Cathepsin B (CB) or Cathepsin L (CL), overexpression reduces α-syn aggregation and provides potent neuroprotection from α-syn-induced neuron death in vitro and in vivo.

Provided herein are methods for treating a neural disorder associated with protein aggregation in a subject comprising administering to the subject an agent that increases expression or activity of cathepsin-D. Optionally, as discussed in more detail below, the neural disorder associated with protein aggregation is a neurodegenerative disease. Optionally, the neural disorder is associated with aggregation of α-synuclein. As used herein, the terms α-syn and α-synuclein are used interchangeably. The agent is selected from the group consisting of a nucleic acid, a polypeptide, an immunoglobulin and a small molecule. Optionally, the polypeptide is CD.

Thus, provided for use in the methods and compositions herein are cathepsin D (CD) and fragments, variants or isoforms of CD. There are a variety of sequences that are disclosed on Genbank, at www.pubmed.gov, and these sequences and others are herein incorporated by reference in their entireties as well as for individual subsequences contained therein. For example, the amino acid and nucleic acid sequences of human CD can be found at GenBank Accession Nos. NP 001900.1 and NM_{—}001909.3, respectively. Thus provided are amino acid sequences of CD comprising an amino acid sequence at least about 70-99% (e.g., 70%, 75%, 80%, 85%, 90%, 95%, 98%, 99%) or more identical to the sequence found at the aforementioned GenBank accession numbers. Also provided are nucleic acids encoding CD comprising a nucleotide sequence at least about 70-99% (e.g., 70%, 75%, 80%, 85%, 90%, 95%, 98%, 99%) or more identical to the nucleotide sequence found at the aforementioned GenBank accession numbers or complement thereof.

As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide can contain up to several amino acid residues or more.

As with all peptides, polypeptides, and proteins, it is understood that substitutions in the amino acid sequence of the CD can occur that do not alter the nature or function of the peptides, polypeptides, or proteins. Such substitutions include conservative amino acid substitutions and are discussed in greater detail below.

The polypeptides provided herein have a desired function. The polypeptides as described herein protect neurons from α-syn induced toxicity. In addition, the polypeptides provided herein prevent aggregation of α-syn. Thus, provided is a method for promoting neuroprotection in a subject comprising administering to the subject an agent that increases expression or activity of cathepsin-D. Optionally, the increase in expression or activity of cathepsin-D prevents protein aggregation and/or prevents accumulation of α-synuclein. The polypeptides are tested for their desired activity using the in vivo or in vitro assays described herein, or by analogous methods, after which their therapeutic, diagnostic or other activities are tested according to known testing methods.

The polypeptides described herein can be modified and varied so long as the desired function is maintained. In general, it is understood that one way to define any known variants and derivatives or those that might arise, of the disclosed genes and proteins herein, is through defining the variants and derivatives in terms of identity to specific known sequences. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the stated sequence or the native sequence. Thus, disclosed are nucleic acids encoding variants of CD which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the nucleic acid encoding CD found at the aforementioned GenBank Accession number. Thus, disclosed are amino acid variants of CD which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the amino acid sequence of CD found at the aforementioned GenBank Accession number. Those of skill in the art readily understand how to determine the homology of two proteins or nucleic acids, such as genes. For example, the identity can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

Fragments, variants, or isoforms of CD are provided as long as the fragments retain the ability to reduce α-syn aggregation or reduce or prevent α-syn induced neuron toxicity. It is understood that these terms include functional fragments and functional variants.

The variants are produced by making amino acid substitutions, deletions, and insertions, as well as post-translational modifications. Variations in post-translational modifications can include variations in the type or amount of carbohydrate moieties of the protein core or any fragment or derivative thereof. Variations in amino acid sequence may arise naturally as allelic variations (e.g., due to genetic polymorphism) or may be produced by human intervention (e.g., by mutagenesis of cloned DNA sequences), such as induced point, deletion, insertion and substitution mutants. These modifications can result in changes in the amino acid sequence, provide silent mutations, modify a restriction site, or provide other specific mutations.

Protein variants and derivatives can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions optionally are made in adjacent pairs, i.e. a deletion of 2 residues or insertion of 2 residues. Substitutions, deletions, insertions or any combination thereof may be combined to arrive at a final construct. The mutations must not place the sequence out of reading frame and optionally will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions

| Amino | Substitutions |
|---|---|
| Acid | (others are known in the art) |
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

As used herein, modification with reference to a polynucleotide or polypeptide, refers to a naturally-occurring, synthetic, recombinant, or chemical change or difference in the primary, secondary, or tertiary structure of a polynucleotide or polypeptide, as compared to a reference polynucleotide or polypeptide, respectively (e.g., as compared to a wild-type polynucleotide or polypeptide). Modifications include such changes as, for example, deletions, insertions, or substitutions. Polynucleotides and polypeptides having such mutations can be isolated or generated using methods well known in the art.

Nucleic acids that encode the aforementioned peptide sequences, variants and fragments thereof are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce CD peptides as well as fragments, isoforms, and variants.

The nucleic acid sequences provided herein are examples of the genus of nucleic acids and are not intended to be limiting. Also provided are expression vectors comprising these nucleic acids, wherein the nucleic acids are operably linked to an expression control sequence. Further provided are cultured cells comprising the expression vectors. Such expression vectors and cultured cells can be used to make the provided polypeptides.

There are a variety of molecules disclosed herein that are nucleic acid based, including for example the nucleic acids that encode CD or fragments or variants thereof. There are a number of compositions and methods which can be used to deliver nucleic acids to cells, either in vitro or in vivo via, for example, expression vectors. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Such methods are well known in the art and readily adaptable for use with the compositions and methods described herein. Further, these methods can be used to target certain diseases and cell populations by using the targeting characteristics of the carrier.

As used herein, plasmid or viral vectors are agents that transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also useful herein are any viral families which share the properties of these viruses and which make them suitable for use as vectors. Retroviral vectors, in general, are described by Verma, I. M., Retroviral vectors for gene transfer. In Microbiology-1985, American Society for Microbiology, pp. 229-232, Washington, (1985), which is incorporated by reference herein in its entirety. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang BioTechniques 15:868-872 (1993), which are incorporated by reference herein in their entireties). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma and a number of other tissue sites. Other useful systems include, for example, replicating and host-restricted non-replicating vaccinia virus vectors.

Optionally, the viral vector is a member of the Paramyxoviridae family. Paramyxovirus vectors are known and are described in, for example, U.S. Pat. No. 6,746,860, which is incorporated by reference herein in its entirety. Examples of paramyxovirus vectors include, but are not limited to, Newcastle disease virus vectors, respiratory syncytial virus (RSV) vectors and parainfluenza viral vectors such as, for example, sendai virus vectors. Parainfluenzavirus vectors include human, mouse and bovine parainfluenzavirus vectors. Parainfluenza viral vectors are known and are described in, for example, U.S. Pat. Nos. 7,341,729; and 7,250,171, which are incorporated herein by reference in their entireties. Newcastle disease virus vectors are known and are described in, for example, U.S. Pat. No. 6,451,323 and 6,146,642, which are incorporated herein in their entireties. Respiratory syncytial virus vectors, include live-attenuated RSV vectors. RSV vectors are known and are described in, for example, U.S. Pat. Nos. 7,205,013; 7,041,489; 6,923,971; and 6,830,748, which are incorporated herein by reference in their entireties. Sendai virus vectors are known and are described in, for example, U.S. Pat. No. 7,314,614; 7,241,617; 7,101,685; and 4,554,158, which are incorporated herein by reference in their entireties.

The provided polypeptides or nucleic acids can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004), which is incorporated by reference herein in its entirety.

The provided polypeptides can be delivered by subviral dense bodies (DB). Dense bodies transport proteins into target cells by membrane fusion. Methods for making and using DBs are described in, for example, Pepperl-Klindworth et al., Gene Therapy 10(3):278-84 (2003), which is incorporated by reference herein in its entirety.

The provided polypeptides can be delivered by tegument aggregates. Methods for making and using tegument aggregates are described in International Publication NO. WO 2006/110728, which is incorporated by reference herein in its entirety. Methods of screening for agents that enhance or increase the expression or activity of CD are provided. Thus, provided is a method of screening for agents that increase expression or activity of cathepsin-D comprising contacting a cell with an agent to be tested and determining the level of expression or activity of cathepsin-D. An increase in the expression or activity of cathepsin-D as compared to a control indicates that the agent increases expression or activity of cathepsin-D.

Also provided is a method of screening for agents that increase expression or activity of cathepsin-D in a subject comprising administering an agent to be tested to the subject and determining the level of expression or activity of cathepsin-D in the subject. An increase in the expression or activity of cathepsin-D as compared to a control indicates that the agent increases expression or activity of cathepsin-D.

As used throughout, a control can comprise either a control cell (e.g., a cell before treatment) or a control sample obtained from a subject (e.g., from the same subject before or after the effect of treatment, or from a second subject without a disorder and/or without treatment) or can comprise a known standard. Optionally, the step of determining the level of expression or activity of CD is determined from a biological sample obtained from the subject. The contacting step occurs in vitro or in vivo.

Also provided is a method of screening for neuroprotective agents comprising contacting a cell with an agent to be tested and determining the level of expression or activity of cathepsin-D. An increase in the expression or activity of cathepsin-D as compared to a control indicates that the agent is a neuroprotective agent.

In addition, a method of screening for neuroprotective agents in a subject, comprising, administering an agent to be tested to the subject and determining the level of expression or activity of cathepsin-D in the subject is provided. An increase in the expression or activity of cathepsin-D as compared to a control indicates that the agent is a neuroprotective agent. Optionally, the step of determining the level of expression or activity of CD is determined from a biological sample obtained from the subject.

Optionally, the method of screening for a neuroprotective agent further includes the step of comprising a potential neuroprotective agent to be tested. The contacting step occurs in vitro or in vivo.

As used herein, the terms enhance or increase mean to increase expression, an activity, response, clinical or laboratory sign of a condition or disease, or other biological parameter. This may include, for example, a 10% increase in expression, activity, response, clinical or laboratory sign of a condition or disease as compared to the native or control level. Thus, the increase can be a 10, 20, 30, 40, 50, 60, 70, 80, 90, 100%, or any amount of increase in between as compared to native or control levels.

In the provided screening methods, the cell can be a prokaryotic or an eukaryotic cell that has, optionally, been transfected with a nucleotide sequence encoding CD or a variant or a fragment thereof, operably linked to a promoter. Using DNA recombination techniques, protein encoding DNA sequences can be inserted into an expression vector, downstream from a promoter sequence.

Such methods allow one skilled in the art to select candidate agents that enhance or increase CD expression or activity. Such agents may be useful as active ingredients included in pharmaceutical compositions. Methods for determining whether the candidate agent enhances or increases expression or activation of CD are known. The assay can be, for example, a Northern blot or one of the provided methods described in the examples below.

Provided herein are compositions with the provided polypeptides or nucleic acids and a pharmaceutically acceptable carrier. The compositions can also be administered in vitro or in vivo. These may be targeted to a particular cell type via antibodies, receptors, or receptor ligands.

By pharmaceutically acceptable is meant a material that is not biologically or otherwise undesirable, i.e., the material may be administered to a subject, along with the provided polypeptides or nucleic acids, without causing undesirable biological effects or interacting in a deleterious manner with other components of the pharmaceutical composition in which it is contained. Pharmaceutical carriers are known to those skilled in the art. The carrier would naturally be selected to minimize any degradation of the active ingredient and to minimize any adverse side effects in the subject. Suitable carriers and their formulations are described in Remington: The Science and Practice of Pharmacy, 21^{st}Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. Further carriers include sustained release preparations such as semipermeable matrices of solid hydrophobic polymers containing the polypeptide or nucleic acid, which matrices are in the form of shaped articles, e.g., films, liposomes or microparticles. It will be apparent to those persons skilled in the art that certain carriers may be more preferable depending upon, for instance, the route of administration and concentration of agent being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. A more recently revised approach for parenteral administration involves use of a slow release or sustained release system such that a constant dosage is maintained. See, e.g., U.S. Pat. No. 3,610,795, which is incorporated by reference herein.

Compositions for oral administration include powders or granules, suspensions or solutions in water or non-aqueous media, capsules, sachets, or tablets. Thickeners, flavorings, diluents, emulsifiers, dispersing aids or binders may be desirable.

The compositions are administered in a number of ways depending on whether local or systemic treatment is desired, and on the area to be treated. The compositions are administered via any of several routes of administration, including, topically, orally, parenterally, intravenously, intraperitoneally, intramuscularly, subcutaneously, intracavity, intraventricularly, transdermally, intrahepatically, intracranially, nebulization/inhalation, or by instillation via bronchoscopy.

Administration of the provided compositions to the brain can be intracranial, intraventricularly, subdural, epidural, or intra-cisternal. For example, the provided compositions can be administered by stereotactic delivery. It is also understood that delivery of compositions to the CNS can be by intravascular delivery if the composition is combined with a moiety that allows for crossing of the blood brain barrier and survival in the blood. Thus, agents can be combined that increase the permeability of the blood brain barrier. To ensure that agents cross the blood brain barrier (BBB), they can be formulated, for example, in liposomes. The liposomes may comprise one or more moieties which are selectively transported into specific cells or organs (targeting moieties), thus providing targeted drug delivery. Exemplary targeting moieties include folate, biotin, mannosides, antibodies, surfactant protein A receptor and gp120.

To ensure that agents of the invention cross the BBB, they may also be coupled to a BBB transport vector (see Bickel, et al., Adv. Drug Delivery Reviews, vol. 46, pp. 247-279, 2001). Exemplary transport vectors include cationized albumin or the OX26 monoclonal antibody to the transferrin receptor; these proteins undergo absorptive-mediated and receptor-mediated transcytosis through the BBB, respectively.

Examples of other BBB transport vectors that target receptor-mediated transport systems into the brain include factors such as insulin, insulin-like growth factors (IGF-I, IGF-II), angiotensin II, atrial and brain natriuretic peptide (ANP, BNP), interleukin I (IL-1) and transferrin. Monoclonal antibodies to the receptors which bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized inimunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties such as, for example, glucose; monocarboxylic acids such as, for example, lactic acid; neutral amino acids such as, for example, phenylalanine; amines such as, for example, choline; basic amino acids such as, for example, arginine; nucleosides such as, for example, adenosine; purine bases such as, for example, adenine, and thyroid hormones such as, for example, triiodothyridine. Antibodies to the extracellular domain of nutrient transporters can also be used as transport vectors.

In some cases, the bond linking the agent to the transport vector may be cleaved following transport into the brain in order to liberate the biologically active compound. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

Optimal dosages of compositions depend on a variety of factors. The exact amount required varies from subject to subject, depending on the species, age, weight and general condition of the subject, the severity of the disease being treated, the particular composition used and its mode of administration. Thus, it is not possible to specify an exact amount for every composition. However, an appropriate amount can be determined by one of ordinary skill in the art using only routine experimentation given the guidance provided herein.

Effective dosages and schedules for administering the compositions may be determined empirically, and making such determinations is within the skill in the art. For example, animal models for a variety of protein aggregate disorders can be obtained, for example, from The Jackson Laboratory, 600 Main Street, Bar Harbor, Me. 04609 USA. Alternatively, the CD mouse model provided herein is used. Both direct (histology) and functional measurements (learning ability, memory skills, neurologic scores and the like) can be used to monitor response to therapy. These methods involve the sacrifice of representative animals to evaluate the population, increasing the animal numbers necessary for the experiments.

The dosage ranges for the administration of the compositions are those large enough to produce the desired effect in which the symptoms of the disease are affected. The dosage is not so large as to cause adverse side effects, such as unwanted cross-reactions and anaphylactic reactions. The dosage is adjusted by the individual physician in the event of any counterindications. Doses are administered in one or more dose administrations daily, for one or several days.

The provided compositions may be used alone or in combination with one or more additive compounds or therapeutic agent. The compound or therapeutic agent may be any compound or substance known in the art which may be beneficial to the subject. The second compound may be any compound which is known in the art to treat, prevent, or reduce the symptoms of a neural disorder associated with protein aggregation. Furthermore, the second compound may be any compound of benefit to the subject when administered in combination with the administration of a compound of the disclosure, e.g. a neuroprotective compound. The language in combination with a second compound or therapeutic agent includes co-administration of the compositions, as well as sequential administration. Thus, the second composition or therapeutic agent can be administered prior to, along with or after, the first composition(s).

Therapeutic agents that are administered in combination with the provided compositions may be effective in controlling detrimental protein aggregate deposition either following their entry into the brain (following penetration of the blood brain barrier) or from the periphery. When acting from the periphery, a therapeutic agent may alter the equilibrium of a protein between the brain and the plasma so as to favor the exit of the protein from the brain. An increase in the exit of the protein from the brain would result in a decrease in the protein brain concentration and therefore favor a decrease in protein deposition in aggregates. Alternatively, therapeutic agents that penetrate the blood brain barrier could control deposition by acting directly on brain proteins, for example, by maintaining it in a non-fibrillar form or favoring its clearance from the brain.

Therapeutic agents for use in the provided methods include, but are not limited to, chemotherapeutic agents, anti-inflammatory agents, anti-viral agents, anti-retroviral agents, anti-opportunistic agents, antibiotics, anticonvulsants, immunosuppressive agents, apoptosis-inducing agents, lazaroids, bioenergetics, antipsychotics, N-methyl D-aspartate (NMDA) antagonists, dopamine antagonists, antidepressants, acetylcholinesterase inhibitors, cholinesterase inhibitors, antiglutamatergic agents, dopamine receptors, dopamine agonists, immunoglobulins and pain medications. Thus, the therapeutic agent can be levodopa, carbidopa, benserazide, gingko biloba, qigong tolcapone, entacapone, bromocriptine, pergolide, pramipexole, ropinirole, cabergoline, apomorphine, lisuride, selegiline, rasafiline, quetiapine, rivastagime, tramiprosate, xaliproden, R-flurbiprofen or leuprolide.

Any of the aforementioned treatments can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

As used herein the terms treatment, treat or treating refer to a method of reducing the effects of a disease or condition or one or more symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, the method for treating a protein aggregate disorder is considered to be a treatment if there is at least a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control. It is understood that treatment does not necessarily refer to a cure or complete ablation of the disease, condition or symptoms of the disease or condition.

As used herein, the terms prevent, preventing and prevention of a disease or disorder refers to an action, for example, of administration of a therapeutic agent, that occurs before a subject begins to suffer from one or more symptoms of the disease or disorder, and which inhibits or delays onset of the severity of one or more symptoms of the disease or disorder.

As used herein, subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. As used herein, patient or subject may be used interchangeably and can refer to a subject afflicted with a disease or disorder. The term patient or subject includes human and veterinary subjects.

Neural or neuronal disorders associated with protein aggregation can be treated or prevented using the methods and compositions provided herein. As used herein, neural disorders associated with protein aggregation includes a disease, disorder or condition that is associated with detrimental protein aggregation in a subject. Detrimental protein aggregation is the undesirable and harmful accumulation, oligomerization, fibrillization or aggregation, of two or more, hetero- or homomeric, proteins or peptides. A detrimental protein aggregate may be deposited in bodies, inclusions or plaques, the characteristics of which are often indicative of disease and contain disease-specific proteins. A detrimental protein aggregate is a three dimensional structure that may contain, for example, misfolded protein composed of β-sheets, fibril-like structures and/or highly hydrophobic domains that tend to aggregate and are toxic to cells. Furthermore, a detrimental protein aggregate may be described as amyloid-like, although it does not contain amyloid deposits and is not considered to be associated with an amyloidosis as it does not adhere to the strict definition of amyloid, i.e., it does not display red-green or apple-green birefringence under polarized light following staining with Congo red.

Neural disorders associated with protein aggregation include disorders characterized by α-synuclein aggregation. Neural disorders associated with protein aggregation include, Parkinson's disease, Lewy body dementia, a Lewy body variant of Alzheimer's disease, Gaucher disease, Niemann-Pick disease, CM2 gangliiosidosis, Tay-Sachs disease, Sandhoff disease, metachromatic leukodystrophy and beta-galactosialidosis.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if an polypeptide is disclosed and discussed and a number of modifications that can be made to a number of molecules including the polypeptide are discussed, each and every combination and permutation of the polypeptide and the modifications that are possible are specifically contemplated unless specifically indicated to the contrary. Likewise, any subset or combination of these is also specifically contemplated and disclosed. This concept applies to all aspects of this disclosure including, but not limited to, steps in methods of making and using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular aspects only and is not intended to be limiting.

### EXAMPLES

### Example 1

### Neuroprotective Role of Cathepsin D Against α-Synuclein Pathogenesis

### Materials and Methods

Mice: Littermates from CD+/− breeding were genotype. Wildtype, CD+/− and CD−/− littermates on C57BL6 background were used for all experiments. Mice at p16, p21 and p25 of age were examined, with data presented all from p25.

Immunohistochemistry: Brains were placed in Bouin's fixative overnight at 4° C. followed by paraffin embedding. Five (5) μm thick sections were used for immunohistochemical studies. The following antibodies were used: mouse anti-NeuN (Chemicon, Temecula, Calif.), mouse anti-α-syn (BD Transduction Lab, Lexington, Ky.), sheep anti-α-syn (Chemicon), goat anti-CD (Santa Cruz Biotechnology, Inc., Santa Cruz, Calif.), mouse anti-GFAP (Chemicon), rabbit anti-Ub (Dako, Denmark), mouse anti-Ub (FK2, Biomol International, Inc., Plymouth Meeting, Pa.), mouse anti-Ub (Chemicon, clone Ubi-1), mouse anti-Ub (Zymed, San Francisco, Calif.), mouse anti-synaptophysin (Chemicon), goat anti-cathepsin B (Santa Cruz Biotechnology, Inc.), rabbit anti-active caspase 3 (Chemicon), and rabbit-anti-GAPDH (Cell Signaling Technology, Danvers, Mass.). Horseradish peroxidase conjugated donkey derived secondary antibodies were used at 1:2000 (Jackson ImmunoResearch, West Grove, Pa.). The sections were then incubated with TSA plus (PerkinElmer, Waltham, Mass.) detection solution with Cy3 or fluorescein tyramide, followed by bisbenzimide staining of nuclear DNA. For chromogenic immunohistochemistry, the sections were incubated with a biotinylated secondary antibody (Vector Laboratories, Burlingame, Calif.) followed by ABC reagent (ABC kit, Vector Laboratories). The immunoreaction was visualized by treating the sections in 0.05% diaminobenzidine (DAB) with hydrogen peroxide. The images were taken using a Leica TCS SP5 confocol microscope or a Zeiss Axiocam CCD camera on a 100W Axioscope bright field and fluorescence microscope.

α-syn aggregation assay: The in vitro system developed by McLean and colleagues was used in which an α-syn-green fluorescent protein (GFP) fusion protein (α-syn-GFP) becomes truncated at the C-terminus, cleaving off GFP, to form visible aggregates in cells when co-expressed with synphilin (McLean et al., Neuroscience 104:901-12 (2001)). H4 neuroglioma cells were transfected with α-syn-GFP and synphilin, and either the empty vector pcDNA3.1 or CD. Twenty-four (24) hours after transfection, cells were fixed and stained with a mouse monoclonal antibody against α-syn (1:1000; BD Transduction Lab, Lexington, Ky.), and a secondary Alexa 488-conjugated goat anti-mouse antibody (1:500; Jackson ImmunoResearch, West Grove, Pa.). An observer blind to the transfection conditions scored neurons as positive or negative for α-syn aggregates visible with a 20× objective under a fluorescent microscope. Three independent experiments were carried out with 4 replicates per experiment. Student t-test was used to compare transfection with empty vector versus transfection with CD.

Cell culture and transfection: The human neuroblastoma SHSY5Y cells were transfected in triplicate by vector alone, GFP-α-syn, pCMV-CD, or co-transfected by GFP-α-syn (or A53T, MOP, Y125A mutant α-syn) and pCMV-CD (or mutant CD) by Amaxa method as described by the vendor. Transfection efficiency was ˜80% as assessed by cells with or without GFP. Seventy-two (72) hours after transfection, cells were harvested. For chloroquine (10 μM) treatment, the chemicals were added 48 hours after transfection, cells were harvested 42 hours later. Live cells were counted by trypan blue exclusion. Relative cell survival was calculated as number of live cells after transfection by pCMV-CD and/or GFP-α-syn divided by live cells after transfection by vector alone. Elevated expression of α-syn or cathepsins was confirmed by Western blot analyses using whole cell extracts.

Western blot: Wildtype and CD−/− cortex (n≧3 each genotype) were homogenized in 10 volumes of ice-cold lyses buffer (50 mM Tris-HCl pH 7.4, 175 mM NaCl, 5 mM EDTA), sonicated for 10 seconds, add TritonX-100 to 1% and incubated for 30 minutes on ice. Homogenates were then centrifuges at 15,000 g for 15 min at 4° C. to separate supernatants (fractions soluble in 1% TritonX-100) and pellets (TritonX-100-insoluble fractions) as described in Giasson et al., Neuron 34:521-33 (2002). Pellets were resuspended in lyses buffer containing 2% SDS. Western blotting for each sample was done at least twice. The antibodies used were described above.

Quantitative PCR: Total brain RNA was isolated from p25 mice using RNA-STAT60 (Tel-Test, Friendswood, Tex.). Total RNA (2 μg) was then reverse transcribed using Applied Biosystems GENEAMP® Gold RNA PCR Reagent Kit (Foster City, Calif.). Real-time PCR reactions were setup in duplicate using TAQMAN® gene assays (Applied Biosystems, Foster City, Calif.) and amplified in an Applied Biosystems Step-One instrument (Foster City, Calif.). ΔΔCCT curves were generated using 18S TAQMAN® gene assays (Applied Biosystems, Foster City, Calif.) as internal standards. Quantitative PCR results are shown as standard deviation of from 3 different amplifications from RNA reverse transcribed from 3 different animals. Individual gene assay kits were purchased from Applied Biosystems for each of the RNAs analyzed. Paired t-tests were conducted on RQ values for each group to determine significance.

Proteasome activity assays: The proteasome activities were analyzed using the Triton-X-100-soluble fractions. The assay buffer consists of 50 mM Tris (pH7.5), 2.5 mM EGTA, 20% glycerol, 1 mM DTT, 0.05% NP-40, 50 μM substrate. Lactacystin was used at a final concentration of 10 μM to block proteasome activities as negative controls. Fluorescence was read at 5 min intervals for 2 hours, at an excitation wavelength of 380 nm and an emission wavelength of 460 nM. Assays were done in triplicate, each using n≧3 mice per genotype.

C. elegans Experiments: Nematodes were maintained following standard procedures (Brenner, Genetics 77:71-94 (1974). Worms expressing α-syn alone UA49 [baInl2; P_{unc-54}::α-syn::gffi, rol-6 (su1006)] or with tor-2 [UA50; baInl3; P_{unc-54}::α-syn::gfr, P_{unc-54}::tor-2, rol-6 (su1006)] were created, integrated into the genome to generate an isogenic line, and out-crossed four times. The worm line was used that overexpresses TOR-2 protein (a worm homolog of human torsinA) and α-syn fused to GFP in the body wall muscle cells because these cells are much larger than neurons for detecting α-syn aggregation. C. elegans dopaminergic neurons have been shown to be refractory to RNAi. Using this isogenic line, the worm CD ortholog was knocked down by RNAi, and scored for the return of α-syn aggregates over the course of development and aging.

RNAi was performed by bacterial feeding as described (Kamath and Ahringer, Methods 30:313-21 (2003)) with the following modification. A CD-specific RNAi feeding clone targeting a distinct portion of the C. elegans open reading frame [R12H7.2 (asp-4); e-value=1.8e-10⁸] with highest homology to human CD (Geneservice, Cambridge, UK) was grown for 14 hours in LB broth with 100 μg/ml ampicillin and seeded onto NGM agar plates containing 1 mM isopropyl 13-D-thiogalactoside. After 4 hours incubation at 25° C. to dry the plates, five gravid adults were then placed onto the corresponding RNAi plates and allowed to lay eggs for 9 hours; the resulting age-synchronized worms were analyzed at the indicated stage. RNAi knockdown was performed in duplicate sets of animals and enhancement α-syn misfolding was scored as positive if at least 80% of worms displayed an increased quantity and size of α-syn::GFP aggregates. For each trial, 20 worms were transferred onto a 2% agarose pad, immobilized with 2 mM levamisole, and analyzed using Nikon Eclipse E800 epifluorescence microscope equipped with Endow GFP HYQ filter cube (Chroma Technology, Rockingham, Vt.). Images were captured with a Cool Snap CCD camera (Photometrics, Tucson, Ariz.) driven by MetaMorph software (Universal Imaging, West Chester, Pa.).

Semi-quantitative RT-PCR. The procedure for total RNA isolation, cDNA preparation, and semi-quantitative RT-PCR was described previously (Hamamichi et al., PNAS 105(2):728-33, 2008). The following primers were used for the PCR:

| cdk-5 | |
|---|---|
| (SEQ ID NO: 1) | |
| Primer 1: | 5′ GGGGATGATGAGGGTGTTCCAAGC 3′ |
| (SEQ ID NO: 2) | |
| Primer 2: | 5′ GGCGACCGGCATTTGAGATCTCTGC 3′ |
| tor-2 | |
| (SEQ ID NO: 3) | |
| Primer 1: | 5′ CAATTATCATGCGTTATACAAAG 3′ |
| (SEQ ID NO: 4) | |
| Primer 2: | 5′ CATTCCACTTCGATAAGTATTG 3′ |

For the DA neurodegeneration analysis, strain UA54 [baEx45; P_{dat-1}::α-syn, Pd_{dat-1}::gfp, P_{dat-1} j::CD, rol-6 (su1006)], UA90 [baEx69; P_{dat-1}::α-syn, P_{dat-1}j::CD D295R, rol-6 (su1006)], UA91 [baEx70; P_{dat-1}::α-syn, P_{dat-1}::CD F2291, rol-6 (su1006)], UA53 [baEx44; P_{dat-1}::α-syn, rol-6 (su1006)], and UA55 [baEx46; P_{dat-1}::α-syn, P_{dat-1}::CL, rol-6 (su1006)] were generated by injecting 50 μg/ml of expression plasmid containing the human cathepsin cDNA and 50 μg/ml of rol-6 into an integrated line of UA44 [baInl1; P_{dat-1}::α-syn, (Cao et al., J. Neurosci. 25:3801-12 (2005))]. Three stable lines were randomly selected for neurodegeneration analysis. The 6 anterior DA neurons (4 CEP and 2 ADE neurons) of 30 animals/trial were examined for neurodegeneration when the animals were 7 days old. 90 animals from each of three CD (or CD D295R, CD F229I, CB, and CL) transgenic lines were analyzed (3 lines×3 trials of 30 animals/trial=270 total animals scored). Worms displaying at least one degenerative change (dendrite, axon, or cell body loss) were scored as exhibiting degenerating neurons as previously reported (Cooper et al., Neuron 313:324-8 (2006); Cao et al., J. Neurosci. 25:3801-12 (2005)).

Results

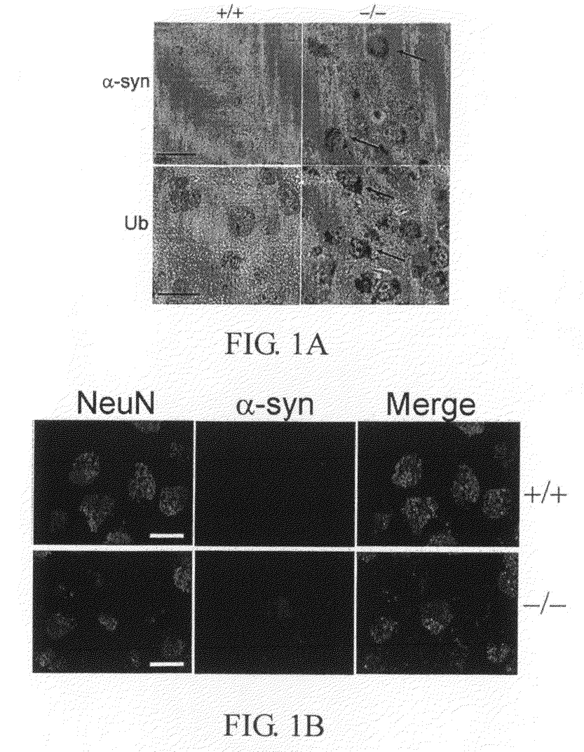

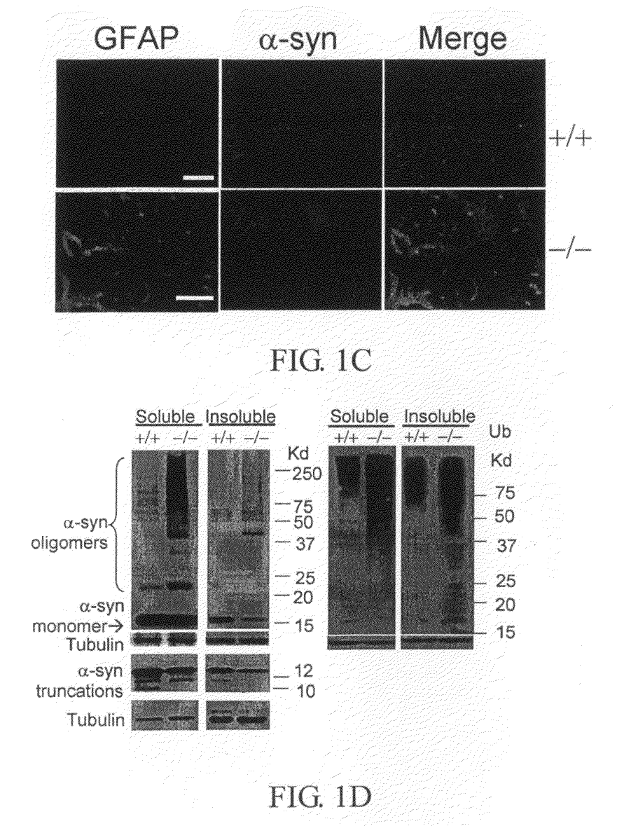

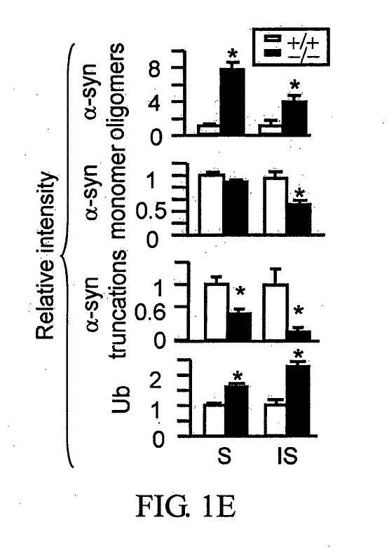

CD deficient mice exhibit extensive aggregation of α-syn in neurons. To investigate the involvement of lysosomal functions in α-syn clearance, mice were analyzed that were deficient in CD, previously generated by a targeted insertion of the neo marker in exon 4 (Saftig et al., EMBO J. 14:3599-3608 (1995)). CD is the main lysosomal aspartyl protease with endopeptidase activity, responsible for rapid turnover of long-lived proteins within the lysosomes, and can cleave α-syn in vitro. CD-deficient (CD−/−) mice die at approximately postnatal day 26 (p26) secondary to a combination of nervous system and systemic abnormalities. Extensive neuron death resulting from activation of both apoptotic and non-apoptotic pathways has been observed in these mice. We examined brains from p21 and p25 CD−/− mice and found significant α-syn aggregation in p25 CD−/− but not wildtype cortex (FIG. 1A). In contrast to the brains of human lipidoses patients where α-syn aggregates in both neurons and glia and co-localizes with lipids, in CD−/− brains α-syn aggregates do not co-localize with autofluorescent lipofuscin. Furthermore, α-syn aggregates in CD−/− brains were present in cells co-expressing the neuron marker NeuN, but not in GFAP-immunoreactive astrocytes (FIGS. 1B and 1C). Accumulation of ubiquitinated proteins also occurs in CD−/− cortex compared to wildtype cortex (FIG. 1A). Consistent with the immunohistochemical studies, we found elevated levels of high molecular weight but not monomeric α-syn, and high molecular weight ubiquitinated proteins in both TritonX100 soluble and insoluble extracts from the cortex of CD−/− mice by western blot analyses, similar to what occurs in LB diseases (FIGS. 1D and 1E). Truncated 12 kd and 10 kd α-syn fragments are reduced in CD−/− extracts (FIGS. 1D and 1E). The cytoplasmic microtubule associated protein, tau, or the synaptic protein, synaptophysin, did not aggregate in CD−/− cortex at p25 compared to wildtype cortex at p25, suggesting that CD deficiency does not have a general effect on the aggregation of all cytoplasmic and synaptic proteins.

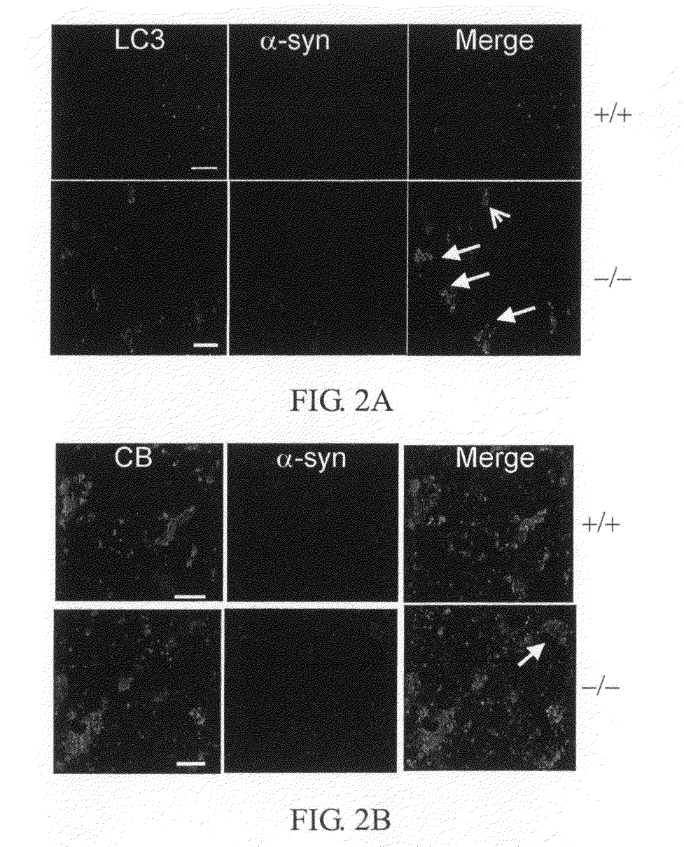

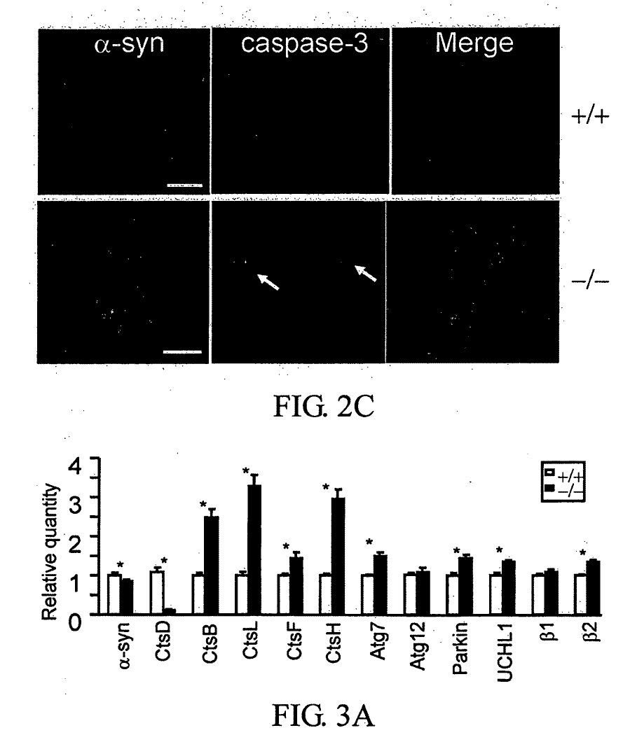

α-syn aggregates are outside of autophagosomes and lysosomes in inflicted neurons. Prior studies found autophagosomes start to accumulate in CD−/− brains as early as p1, compared to CD+/+age-matched controls (Koike et al., J. Neurosci. 20:6898-6906 (2000)). Furthermore, CB immunostaining as well as enzymatic activities are increased as early as p21 in CD−/− brains (Koike et al., J. Neurosci. 20:6898-6906 (2000)). Electron microscopy studies demonstrated that CB was associated with irregularly shaped and membrane-bound structures containing electron-dense materials, characterizing them as lysosomes in CD−/− brains (Koike et al., J. Neurosci. 20:6898-6906 (2000)). As described herein, it was observed that α-syn aggregation does not become prominent until near p25 in CD−/− brains. α-syn aggregation does not occur in every neuron that exhibits enhanced LC3 staining, indicating that AV accumulation precedes α-syn aggregation (FIG. 2A). It was also observed that α-syn aggregates are adjacent to, but do not overlap with, ATG8/LC3 or CB, suggesting that the aggregates formed outside of autophagosomes and lysosomes (FIG. 2B). Neuronal populations immunoreactive for the apoptotic marker, cleaved caspase-3, are distinct from those with intense α-syn aggregates (FIG. 2C).

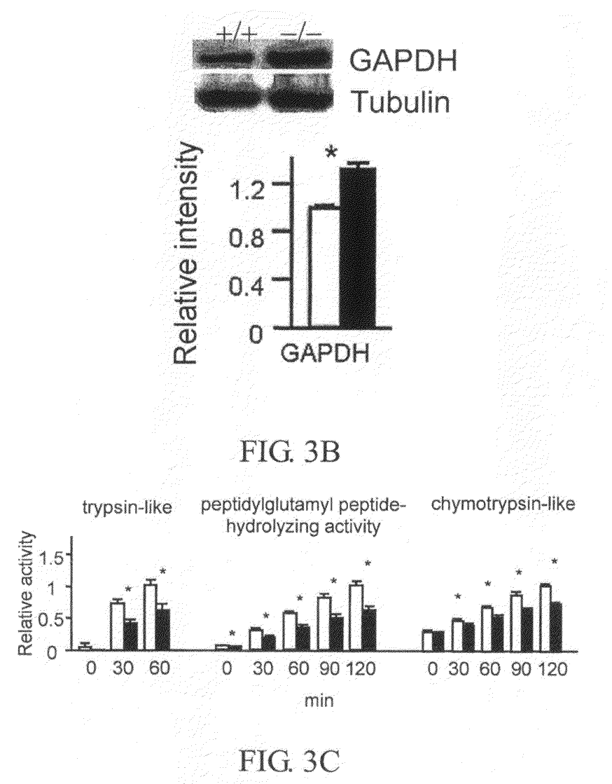

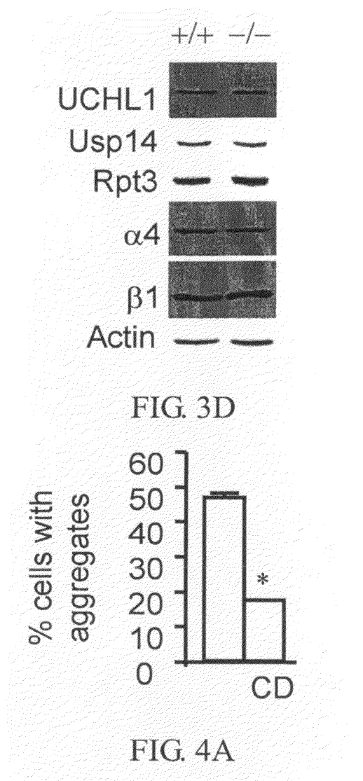

α-syn aggregation is not due to up-regulation of its mRNA, and appears despite of compensatory up-regulation of other proteases. While bulk protein degradation appears to be normal in CD−/− mice (Saftig et al., EMBO J. 14:3599-3608 (1995)), as described herein, it was observed that α-syn mRNA is down-regulated in CD−/− brains at p25 when α-syn aggregation occurs (FIG. 3A). This is consistent with the finding that α-syn mRNA is either unchanged or down-regulated in the majority of sporadic PD cases (Cantuti-Castelvetri et al., J. Neuropathol. Exp. Neurol. 64:1058-1066 (2005)), further showing that α-syn aggregation is likely to be the consequence of deficient protein degradation rather than elevated gene expression.

Prior studies reported that CB but not CL protein is up-regulated in CD−/− brains at p23 (Koike et al., J. Neurosci. 20:6898-6906 (2000)). To better understand the role of CD in selective protein degradation and PD, the expression of genes encoding other brain-enriched lysosomal proteases, autophagy factors, proteasome subunits, and genes linked to familial PD was analyzed. Interestingly, CB, CL, CF, and CH mRNAs are all up-regulated at p25 (FIG. 3A). This result suggests a common transcription regulatory mechanism for these cathepsins in response to CD deficiency. Alternatively, the influx of macrophages or microglia into the CD−/− brain at this age may lead to an increase in cathepsin mRNA expression (Nakanishi et al., J. Neurosci. 21:7526-7533 (2001)). In addition, up-regulation of these protein products may or may not be prominent until p25. We also determined that accumulation of autophagosomes in CD−/− neurons is accompanied with transcription up-regulation of Atg7 but not Atg12 (FIG. 3A). Up-regulation of Atg7 may indicate an increase of autophagosome production in addition to a blockade of autophagy completion. Parkin, UCHL1, and UPS β2 subunit mRNA are also modestly up-regulated in response to CD deficiency, indicating significant compensatory response to CD deficiency at the level of gene transcription (FIG. 3A).

CD deficiency reduces proteasome activities. In addition to deficient macroautophagy, an accumulation of GAPDH, a substrate of CMA, was observed (FIG. 3B). Reduced proteasome activity in CD−/− brain extracts was also observed (FIG. 3C), suggesting a functional interaction between the two major α-syn clearance machineries, lysosomes and proteasomes. Accumulation of ubiquitinated proteins appears at p21, when proteasome activities are largely unaltered, compared to that in wildtype brains. None of the proteins examined by western analyses were significantly changed in CD−/− brains, including UCHL1, a gene mutated in familial PD and a ubiquitin hydrolase and E3 ligase; Usp14, a key deubiquitination enzyme; Rpt3, an ATPase regulatory subunit, a4 subunit that is important for the gating into the 20S core particle, and b1 subunit that is part of the proteasome core (FIG. 3D).

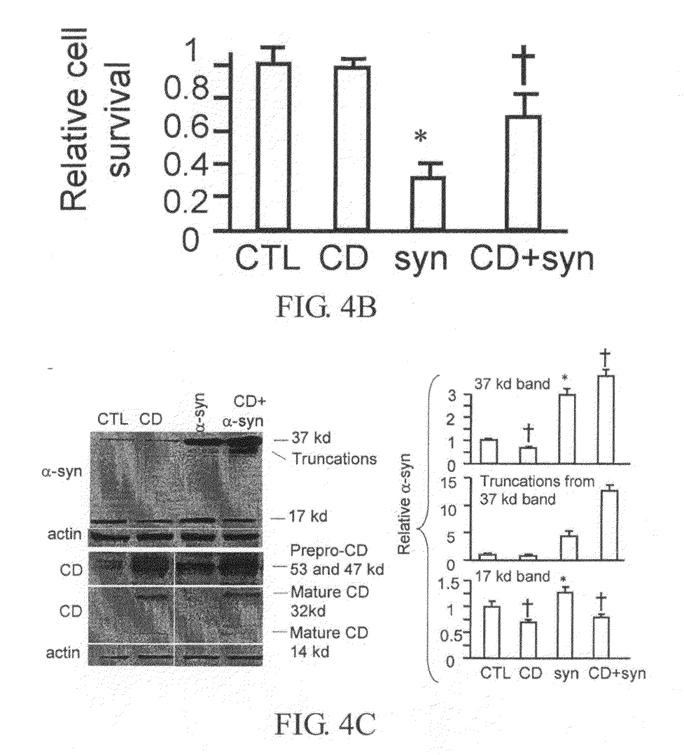

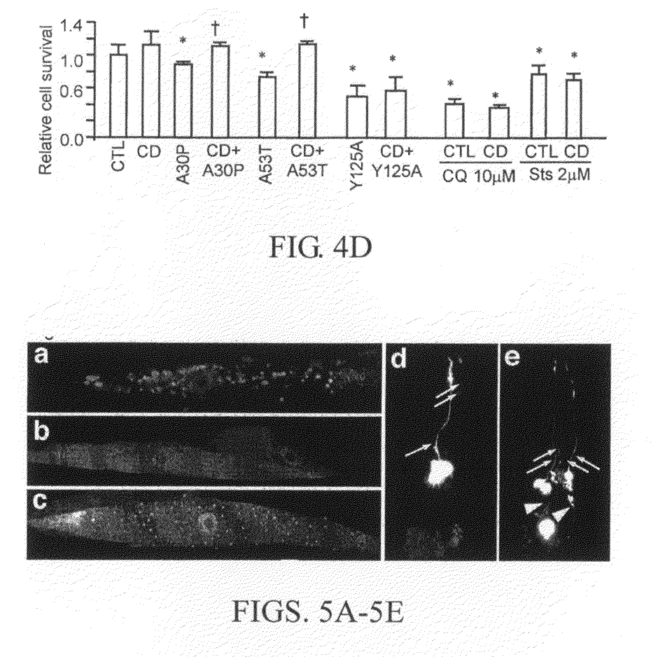

Overexpression of CD reduces α-syn aggregation in mammalian cells. To further understand how CD activity influences α-syn homeostasis, the hypothesis that enhancing CD expression can reduce α-syn aggregation was tested. The simple culture system developed by McLean and colleagues was used in which an α-syn-green fluorescent protein (GFP) fusion protein (α-syn-GFP) forms visible aggregates in cells when co-expressed with synphilin (McLean et al., Neuroscience 104:901-12 (2001)). H4 neuroglioma cells were transfected with α-syn-GFP, synphilin, and CD. As a control, cells were transfected with α-syn-GFP, synphilin and empty vector pcDNA3.1. Approximately 50% of control transfected cells exhibited α-syn aggregates. Remarkably, transfection of CD together with α-syn-GFP and synphilin led to less than 20% of transfected cells exhibiting α-syn aggregates (FIG. 4A).

Overexpression of CD is neuroprotective against α-syn toxicity in mammalian cells. Excessive α-syn induces neuron death in cell cultures, and in a variety of genetic and viral delivery based animal models. To examine the potential of elevating CD level as a means to reduce α-syn-induced cell death, human neuroblastoma SHSY5Y cells were transfected with α-syn, in the absence or presence of increased CD expression (FIG. 4B). Similar to previous studies of α-syn overexpression in yeast, worms and rat neurons, it was observed that overexpression of wildtype α-syn induced robust cell death in SHSY5Y cells. Co-transfection of the human CD provided significant protection against α-syn overexpression-induced cell death (FIG. 4B). Furthermore, co-expression of CD with α-syn-GFP in human neuroblastoma SHSY5Y cells produced a cleavage product of α-syn-GFP and reduced endogenous monomeric 17 kd α-syn (FIG. 4C).

α-syn point mutation at the major CD cleavage site results in resistance to CD neuroprotection. α-syn is rich in hydrophobic amino acids (52%) and is natively unfolded. CD has a known specificity in recognizing hydrophobic residues. Although α-syn contains many putative CD cleavage sites, the main cleavage occurs at A124-Y125 (Hossain et al., J. Alzheimers Dis. 3:577-584 (2001)). It was observed that CD is also protective against PD-causing mutant α-syn induced-cell death in SHSY5Y cells (FIG. 4D). In contrast, mutating α-syn at the putative CD cleavage site Y125 results in an α-syn mutant that induces cell death that resists neuroprotection by elevated CD (FIG. 4D). Furthermore, CD is ineffective at attenuating chloroquine- or staurorosporine-induced cell death (FIG. 4D).

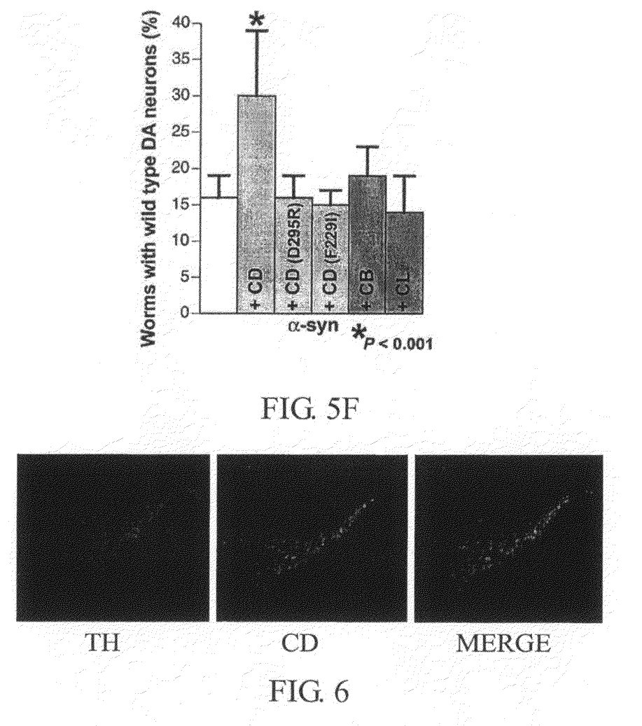

Overexpression of CD is neuroprotective in C. elegans. To further investigate the role of CD activity in α-syn clearance in vivo, transgenic C. elegans were generated expressing a human α-syn and GFP fusion protein in body wall muscle cells. In these worms, human α-syn::GFP forms aggregates as worms develop and age (FIG. 5A). Co-expression of the worm TOR-2 protein chaperone ameliorated the formation of α-syn::GFP aggregates (FIG. 5b). Importantly, this established a genetic background within which enhancement of α-syn aggregation could be more readily visualized by RNA interference (RNAi). Using bacterial RNAi feeding to specifically target the C. elegans ortholog of CD, CD was knocked down in α-syn::GFP+TOR-2 transgenic worms. RNAi targeting of CD led to a return of fluorescent aggregates over time (FIG. 5c). Taken together, CD deficiency led to α-syn aggregation in both mice and worms.

It was further investigated whether human CD attenuates the loss of dopaminergic neurons in a C. elegans model of α-syn-induced neurodegeneration (Cooper et al., Science 313:324-8 (2006)). Overexpression of α-syn led to dopaminergic neuron death, as evidenced by the finding that only 16% of 7 d old adult α-syn expressing worms (n=270 worms analyzed) displayed normal numbers of dopaminergic neurons (FIGS. 5D and 5F). In contrast, co-overexpression of human CD significantly protected against dopaminergic neurodegeneration, since 30% of same-staged animals (n=270) exhibited wild-type dopaminergic neurons (FIGS. 5E and 5F; p<0.001, Fisher Exact Test). Overexpression of enzymatic mutants of CD (D295R and F229I) (17; 40), or related human cathepsin gene products, CB or CL, did not attenuate dopaminergic neuron death in this in vivo assay (FIG. 5F), thereby suggesting a specific role of CD in neuroprotection against α-syn-induced cell death, as well as an essential role of CD enzymatic activity in this neuroprotection.

### Example 2

### CD Expression in Mouse Substantia Nigra-Pars Reticulata (SNr)

A CD expressing construct using the rAAV-CBA-IRES-EGFP-WPRE vector was created as described, which co-expresses CD and enhanced green fluorescent protein (EGFP) under CMV promoter (St. Martin et al., J. Neurochem. 100(6):1449-57 (2007)). AAV-CD was injected using stereotaxic method in unilateral SNr region at 3 months of age as described previously (St. Martin et al., J. Neurochem. 100(6):1449-57 (2007)). Three (3) microliters of 8.2×10¹⁰ vg/microliter was injected into the mice. One month after injection, the mice were perfused and immunohistochemistry studies were performed for the expression and localization of TH+neurons and CD. FIG. 6 shows that AAV-CD delivered and allowed expression of CD in the SNr of mice.

1. A method of screening for an agent that increases expression or activity of cathepsin-D comprising,
a) contacting a cell with an agent to be tested; and
b) determining the level of expression or activity of cathepsin-D, wherein an increase in the expression or activity of cathepsin-D as compared to a control indicates that the agent increases expression or activity of cathepsin-D.

2. A method of screening for an agent that increases expression or activity of cathepsin-D in a subject comprising,
a) administering an agent to be tested to the subject; and
b) determining the level of expression or activity of cathepsin-D in the subject, wherein an increase in the expression or activity of cathepsin-D as compared to a control indicates that the agent increases expression or activity of cathepsin-D.

3. A method of screening for a neuroprotective agent, comprising,
a) contacting a cell with an agent to be tested; and
b) determining the level of expression or activity of cathepsin-D, wherein an increase in the expression or activity of cathepsin-D as compared to a control indicates that the agent is a neuroprotective agent.

4. The method of claim 3, further comprising selecting a potential neuroprotective agent to be tested.

5. A method of screening for a neuroprotective agent in a subject, comprising,
a) administering an agent to be tested to the subject; and
b) determining the level of expression or activity of cathepsin-D in the subject, wherein an increase in the expression or activity of cathepsin-D as compared to a control indicates that the agent is a neuroprotective agent.

6. The method of claim 5, further comprising selecting a potential neuroprotective agent to be tested.

7. The method of claim 1, wherein the contacting step is in vitro.

8. The method of claim 1, wherein the contacting step is in vivo.

9. The method of claim 2, wherein step (b) is determined from a biological sample obtained from the subject.

10. A method for promoting neuroprotection in a subject comprising administering to the subject an agent that increases expression or activity of cathepsin-D.

11. The method of claim 10, wherein an agent identified by the method of any one of claims 1 to 7 is administered to the subject.

12. The method of claim 10, wherein the increase in expression or activity of cathepsin-D prevents protein aggregation.

13. The method of claim 10, wherein the increase in expression or activity of cathepsin-D prevents accumulation of α-synuclein.

14. A method for treating a neural disorder associated with protein aggregation in a subject comprising administering to the subject an agent that increases expression or activity of cathepsin-D.

15. The method of claim 14, wherein the neural disorder associated with protein aggregation is a neurodegenerative disease.

16. The method of claim 14, wherein the neural disorder is associated with aggregation of α-synuclein.

17. The method of claim 14, wherein the neural disorder associated with protein aggregation is selected from the group consisting of Parkinson's disease, Lewy body dementia and a Lewy body variant of Alzheimer's disease.

18. The method of claim 14, wherein the agent is selected from the group consisting of a nucleic acid, a peptide, a protein, an immunoglobulin and a small molecule.

19. The method of claim 18, wherein the protein is a cathepsin-D.

20. The method of claim 18, wherein the peptide is a peptide with 80 to 100% sequence similarity to cathepsin-D.

21. The method of claim 18, wherein the nucleic acid is a nucleic acid that encodes cathepsin-D.

22. The method of claim 21, wherein a vector comprises the nucleic acid that encodes cathepsin-D.

23. The method of claim 22, wherein the vector is a plasmid or viral vector.

24. The method of claim 21, wherein the nucleic acid encoding cathepsin-D is operably linked to a promoter.

25. The method of claim 3, wherein the contacting step is in vitro.

26. The method of claim 3, wherein the contacting step is in vivo.

27. The method of claim 5, wherein step (b) is determined from a biological sample obtained from the subject.

28. The method of claim 10, wherein the agent is selected from the group consisting of a nucleic acid, a peptide, a protein, an immunoglobulin and a small molecule.

29. The method of claim 28, wherein the protein is a cathepsin-D.

30. The method of claim 28, wherein the peptide is a peptide with 80 to 100% sequence similarity to cathepsin-D.

31. The method of claim 28, wherein the nucleic acid is a nucleic acid that encodes cathepsin-D.

32. The method of claim 31, wherein a vector comprises the nucleic acid that encodes cathepsin-D.

33. The method of claim 32, wherein the vector is a plasmid or viral vector.

34. The method of claim 33, wherein the nucleic acid encoding cathepsin-D is operably linked to a promoter.
